(19) United States
(12) Patent Application Publication
(10) Pub. No.: US 2025/0040918 A1
(43) Pub. Date: Feb. 6, 2025

(54) SAMPLE CONTAINER AND COAXIAL INTRODUCER CANNULA FOR A BIOPSY APPARATUS

(71) Applicant: C. R. Bard, Inc., Franklin Lakes, NJ (US)

(72) Inventors: Angela K. Jensen, Phoenix, AZ (US); Søren F. Ørts, Virum (DK); Jens J. Holme, Lyngby (DK)

(73) Assignee: C. R. Bard, Inc., Franklin Lakes, NJ (US)

(21) Appl. No.: 18/908,884

(22) Filed: Oct. 8, 2024

Related U.S. Application Data
(62) Division of application No. 16/764,966, filed on May 18, 2020, now Pat. No. 12,137,887.
(60) Provisional application No. 62/592,641, filed on Nov. 30, 2017.
(60) Provisional application No. 62/669,015, filed on May 9, 2018.

(51) Int. Cl. A61B 10/02; A61B 10/00

(52) U.S. Cl. CPC A61B 10/0283; A61B 10/0096; A61B 10/0275; A61B 2010/0208

(57) ABSTRACT

A coaxial introducer cannula is provided for use with a biopsy apparatus. The coaxial introducer cannula may comprise a coaxial cannula and a hub. The hub may be fixedly attached to a proximal portion of the coaxial cannula. The hub may have a hub body, a latching lever, and a latch. The latch may be configured to rotatably engage a catch of the biopsy apparatus, and the latching lever may extend radially from the hub body. The latching lever may be longer than a height of a front plate of the biopsy apparatus so that the latching lever can be reached and rotationally operated to rotate the hub relative to the front plate of the biopsy apparatus.

### CROSS-REFERENCE TO RELATED APPLICATIONS

This application is a divisional application of U.S. patent application Ser. No. 16/764,966 entitled “SAMPLE CONTAINER AND COAXIAL INTRODUCER CANNULA FOR A BIOPSY APPARATUS” filed Nov. 29, 2018, which is a national stage entry of international patent application PCT/US2018/063055 entitled “SAMPLE CONTAINER AND COAXIAL INTRODUCER CANNULA FOR A BIOPSY APPARATUS” filed Nov. 29, 2018, which claims priority to U.S. provisional patent application Ser. No. 62/592,641 entitled “SAMPLE CONTAINER AND COAXIAL CANNULA FOR BIOPSY APPARATUS” filed Nov. 30, 2017, and U.S. provisional patent application Ser. No. 62/669,015 entitled “SAMPLE CONTAINER FOR A SIMS BIOPSY DEVICE” filed May 9, 2018, which are incorporated herein by reference in their entireties.

### TECHNICAL FIELD

The present invention relates to a biopsy apparatus, and, more particularly, to a biopsy apparatus having a coaxial introducer cannula and/or sample container.

### BACKGROUND ART

A biopsy may be performed on a patient to help in determining whether the tissue in a region of interest includes cancerous cells. One biopsy technique used to evaluate breast tissue, for example, involves inserting a biopsy probe into the breast tissue region of interest to capture one or more tissue samples from the region. Such a biopsy technique often utilizes a vacuum to pull the tissue to be sampled into a sample notch of the biopsy probe, after which the tissue is severed and collected in a sample container. Efforts continue in the art to improve the ability for a practitioner to access a biopsy site, and/or to transport the severed tissue sample to a sample container for collecting severed tissue samples.

For example, after performing a biopsy under ultrasound needle guidance, some operators may prefer not to handle the tissue specimens, and rather, may prefer to place the tissue specimens directly into formalin, which is a preservative for biological specimens. In contrast, other operators may require access to the tissue specimens for examination prior to placing in formalin. Also, when removing specimens with current devices, such as an open specimen tray, there may be a risk of specimen loss. In addition, anecdotal reports from physicians, including pathologists, suggest that tissue handling by the operators (physicians and technologists) during and after a biopsy procedure could impact the quality of the specimens for pathological analysis.

In addition, some operators may prefer to use a coaxial cannula to maintain an access path to the lesion when utilizing a biopsy apparatus, such as a biopsy driver coupled to a biopsy probe, or a trocar. In one such procedure, the operator may want to place a tissue marker at the biopsy site, in which case the biopsy probe may be withdrawn from the coaxial introducer cannula, and a marker applicator may be inserted through the coaxial introducer cannula to the biopsy site, so as to maintain the lesion/site position after sampling without having to re-target the lesion location using ultrasound. However, the connection and/or disconnection of the coaxial introducer cannula to the biopsy apparatus typically requires the use of two hands, and may be awkward or difficult.

What is needed in the art is a sample container for a biopsy apparatus that facilitates efficient reception and processing of collected tissue samples, and/or a coaxial introducer cannula that facilitates efficient connection and disconnection of the coaxial introducer cannula to the biopsy apparatus.

### SUMMARY OF INVENTION

The present invention provides a sample container for a biopsy apparatus that facilitates efficient reception and processing of collected tissue samples, and/or a coaxial introducer cannula that facilitates efficient connection and disconnection of the coaxial introducer cannula to the biopsy apparatus.

The invention in one form is directed to a biopsy apparatus that includes a biopsy needle, a sample manifold, and a sample container. The sample manifold is coupled to the biopsy needle. The sample manifold has a receptacle and an insertion axis. The sample receptacle has an interior side wall and a mounting pin that projects inwardly from the interior side wall toward the insertion axis. The sample container is configured for insertion into the receptacle. The sample container includes a mounting channel that is sized and positioned to engage and follow the mounting pin of the receptacle as the sample container is rotated.

The invention in another form is directed to a coaxial introducer cannula for use with the biopsy apparatus. The biopsy apparatus has a front plate having a catch, and a biopsy needle extends from the front plate on a longitudinal axis. The coaxial introducer cannula includes a coaxial cannula and a hub. The coaxial cannula is sized to be coaxially and slidably received over the biopsy needle. The hub is fixedly attached to a proximal portion of the coaxial cannula. The hub has a hub body, a latching lever, and a latch. The latch is configured to rotatably engage the catch. The latching lever extends radially from the hub body relative to the longitudinal axis. The latching lever is longer than a height of the front plate so that the latching lever can be reached and rotationally operated to rotate the hub relative to the front plate of the biopsy apparatus, thereby facilitating single-handed rotation of the coaxial introducer cannula relative to the front plate, so as to effect a respective engagement or disengagement of the latch of the coaxial introducer cannula with the catch of the front plate.

One advantage of the sample container aspect of the present invention is that the sample container, in a closed position, may be removed from the biopsy apparatus and placed directly into formalin, without having to open the sample container or handle the tissue samples.

Another advantage of the sample container aspect of the present invention is that it provides an easy-to-use opening mechanism that allows for tissue access, if desired, but also will keep the sample container closed during removal from the biopsy system to help reduce the chance of tissue loss.

One advantage of the coaxial introducer cannula aspect of the present invention is that the operator is able to detach the coaxial introducer cannula from the biopsy apparatus with a single hand, improving the case of use of the coaxial introducer cannula and the accuracy of any subsequent marker placement.

### BRIEF DESCRIPTION OF DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a perspective view of a biopsy apparatus having a sample container in accordance with an aspect of the present invention, and with a biopsy probe assembly attached to a driver assembly;

FIG. 2 is a block representation of the driver assembly of FIG. 1;

FIG. 3A is perspective view of a portion of the biopsy probe assembly of FIG. 1 that includes the sample manifold and a sample container in accordance with an aspect of the present invention;

FIG. 3B is an exploded perspective view of a portion of the biopsy probe assembly of FIG. 1 that includes the sample manifold and sample container, with the sample container in an insertion orientation relative to the sample manifold;

FIG. 3C is a top view of the sample manifold of FIGS. 3A and 3B;

FIG. 4A is perspective view of the sample container of FIGS. 3A and 3B, with the sample container in the closed position;

FIG. 4B is an end view of the sample container of FIGS. 3A and 3B, with the sample container in the closed position;

FIG. 4C is a side view of the sample container of FIGS. 3A and 3B, with the sample container in the closed position;

FIG. 4D is a side view of the sample container of FIGS. 3A and 3B, with the sample container in an intermediate open position;

FIG. 4E is a side view of the sample container of FIGS. 3A and 3B, with the sample container in a fully open position;

FIG. 5 is a perspective view of a portion of the biopsy apparatus of FIG. 1, with a coaxial introducer cannula in accordance with an aspect of the present invention mounted to the biopsy probe assembly of the biopsy apparatus;

FIG. 6A is a perspective view of the coaxial introducer cannula of FIG. 5;

FIG. 6B is an end view of the coaxial introducer cannula of FIG. 5;

FIG. 6C is a side view of the coaxial introducer cannula of FIG. 5;

FIG. 7A is a perspective view of the probe housing of the biopsy apparatus of FIG. 1, with an alternative configuration for connection to a coaxial introducer cannula.

FIG. 7B is an end view of the probe housing of FIG. 7A.

FIG. 8A is a perspective view of an alternative coaxial introducer cannula configured for connection to the alternative connection configuration of the probe housing of FIGS. 7A and 7B;

FIG. 8B is a rear perspective view of the alternative coaxial introducer cannula of FIG. 8A.

FIG. 9 is perspective view of a trocar configured for use with the coaxial introducer cannula of FIGS. 8A and 8B.

Corresponding reference characters indicate corresponding parts throughout the several views. The exemplifications set out herein illustrate at least one embodiment of the invention, and such exemplifications are not to be construed as limiting the scope of the invention in any manner.

### DESCRIPTION OF EMBODIMENTS

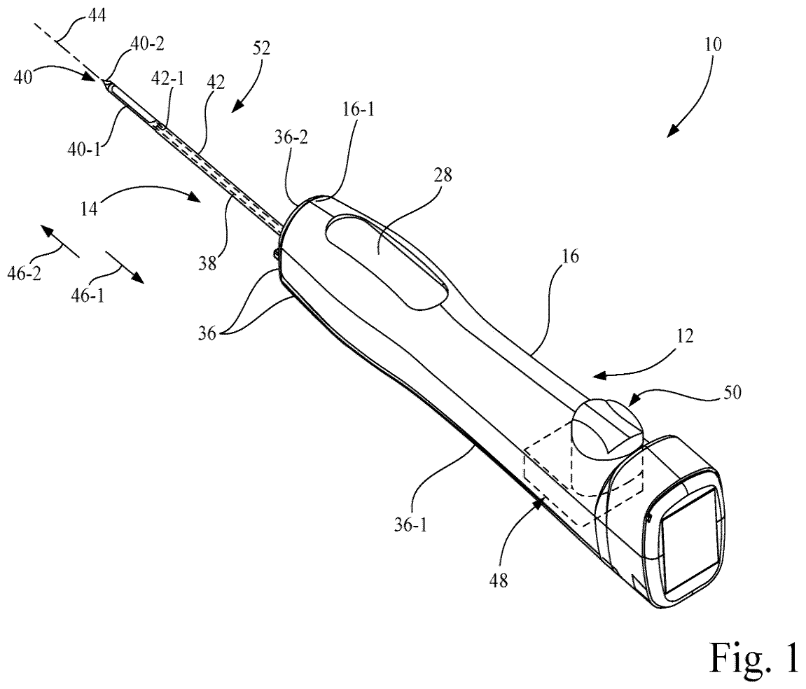

Referring now to the drawings, and more particularly to FIG. 1, there is shown a biopsy apparatus 10 which generally includes a non-invasive, e.g., non-disposable, driver assembly 12 and an invasive, e.g., disposable, biopsy probe assembly 14. As used herein, the term “non-disposable” is used to refer to a device that is intended for use on multiple patients during the lifetime of the device, and the term “disposable” is used to refer to a device that is intended to be disposed of after use on a single patient. Driver assembly 12 includes a driver housing 16 that is configured and ergonomically designed to be grasped by an operator.

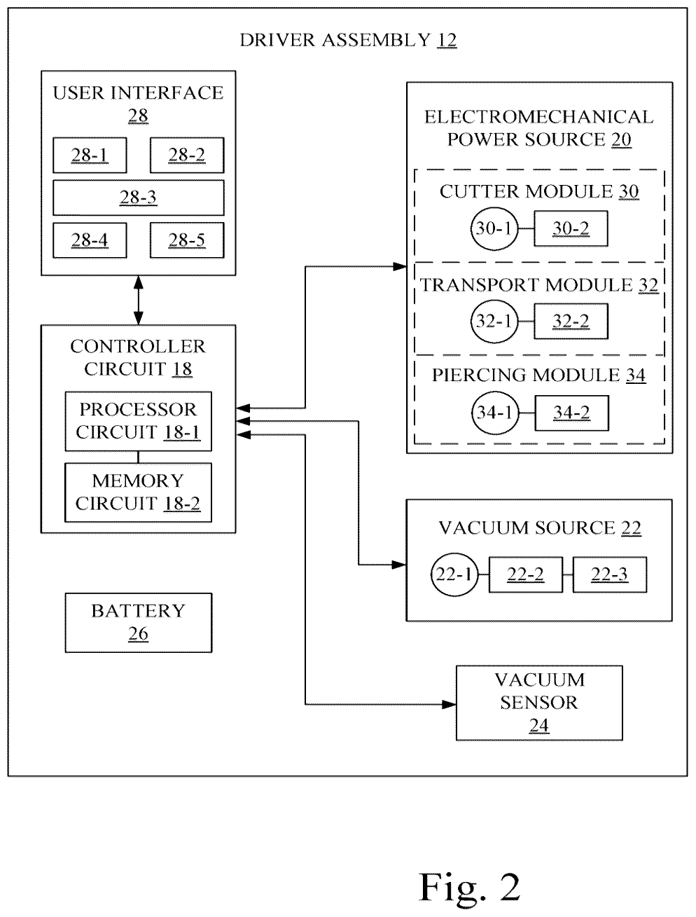

Referring also to FIG. 2, driver assembly 12 includes within driver housing 16 a controller circuit 18, an electromechanical power source 20, a vacuum source 22, a vacuum sensor 24, and a battery 26 (or alternatively an AC adapter). A user interface 28 (see FIG. 1), such as a keypad, is located to be mounted to driver housing 16, and externally accessible by the operator with respect to driver housing 16.

Battery 26 may be, for example, a rechargeable battery, which may be charged by an inductive charging device, or alternatively, by an electrical connection to an electrical power supply. Battery 26 is electrically coupled to controller circuit 18, electromechanical power source 20, vacuum source 22, and user interface 28.

User interface 28 may include control buttons and visual/aural indicators, with the control buttons providing user control over various functions of biopsy apparatus 10, and with the visual/aural indicators providing visual/aural feedback of the status of one or more conditions and/or positions of components of biopsy apparatus 10. The control buttons may include a sample button 28-1 and a prime/pierce button 28-2. The visual indicators may include a display screen 28-3 and/or one or more light emitting diodes (LED) 28-4. The aural indicator may include a buzzer 28-5. The control buttons may include tactile feedback to the operator when activated.

Controller circuit 18 is electrically and communicatively coupled to electromechanical power source 20, vacuum source 22, vacuum sensor 24, and user interface 28, such as by one or more wires or circuit traces. Controller circuit 18 may be assembled on an electrical circuit board, and includes, for example, a processor circuit 18-1 and a memory circuit 18-2.

Processor circuit 18-1 has one or more programmable microprocessors and associated circuitry, such as an input/output interface, clock, buffers, memory, etc. Memory circuit 18-2 is communicatively coupled to processor circuit 18-1, e.g., via a bus circuit, and is a non-transitory electronic memory that may include volatile memory circuits, such as random access memory (RAM), and non-volatile memory circuits, such as read only memory (ROM), electronically erasable programmable ROM (EEPROM), NOR flash memory, NAND flash memory, etc. Controller circuit 18 may be formed as one or more Application Specific Integrated Circuits (ASIC).

Controller circuit 18 is configured via software and/or firmware residing in memory circuit 18-2 to execute program instructions to perform functions associated with the retrieval of biopsy tissue samples, such as that of controlling and/or monitoring one or more components of electromechanical power source 20, vacuum source 22, and vacuum sensor 24.

Electromechanical power source 20 may include, for example, a cutter module 30, a transport module 32, and a piercing module 34, each being respectively electrically coupled to battery 26. Each of cutter module 30, transport module 32, and piercing module 34 is electrically and controllably coupled to controller circuit 18 by one or more electrical conductors, e.g., wires or circuit traces.

Cutter module 30 may include an electrical motor 30-1 having a shaft to which a cutter drive 30-2 is drivably connected. Transport module 32 may include an electrical motor 32-1 having a shaft to which a transport drive 32-2 is drivably connected. Piercing module 34 may include an electrical motor 34-1 having a shaft to which a piercing shot drive 34-2 is drivably connected. Each electrical motor 30-1, 32-1, 34-1 may be, for example, a direct current (DC) motor or stepper motor. Each of cutter drive 30-2, transport drive 32-2, and piercing shot drive 34-2 may include one or more of a gear, gear train, or belt/pulley arrangement.

Vacuum source 22 is electrically and controllably coupled to battery 26 by one or more electrical conductors, e.g., wires or circuit traces. Vacuum source 22 may include, for example, an electric motor 22-1 that drives a vacuum pump 22-2. Vacuum source 22 has a vacuum source port 22-3 coupled to vacuum pump 22-2 for establishing vacuum in biopsy probe assembly 14. Electric motor 22-1 may be, for example, a rotary, linear or vibratory DC motor. Vacuum pump 22-2 may be, for example, a peristaltic pump or a diaphragm pump, or one or more of each connected in series or parallel.

Vacuum sensor 24 is electrically coupled to controller circuit 18 by one or more electrical conductors, e.g., wires or circuit traces. Vacuum sensor 24 may be a pressure differential sensor that provides vacuum (negative pressure) feedback signals to controller circuit 18. In some implementations, vacuum sensor 24 may be incorporated into vacuum source 22.

Referring again to FIG. 1, biopsy probe assembly 14 is configured for releasable attachment to driver assembly 12. As used herein, the term “releasable attachment” means a configuration that facilitates an intended temporary connection followed by selective detachment involving a manipulation of disposable biopsy probe assembly 14 relative to driver assembly 12, without the need for tools.

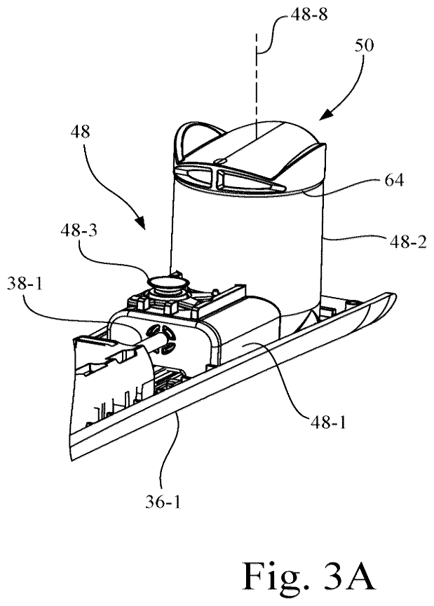

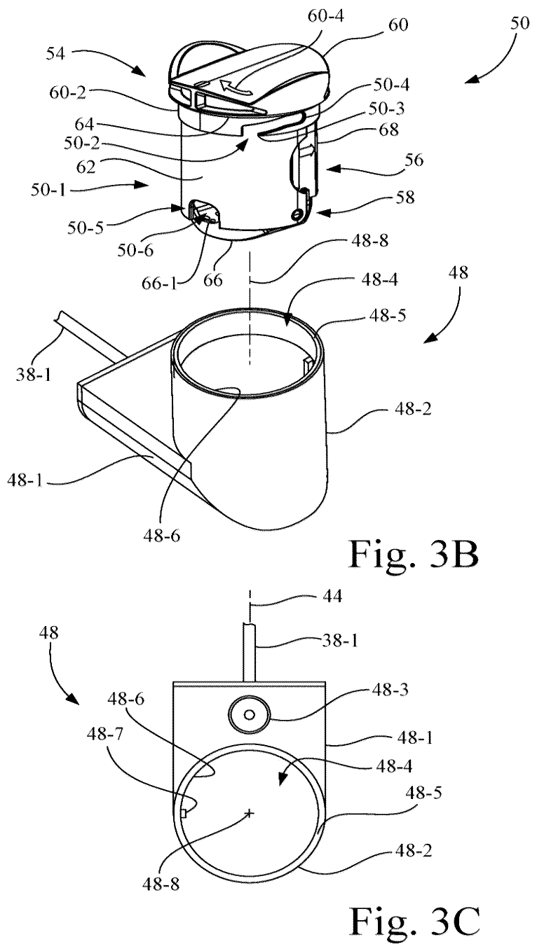

Referring to FIGS. 1 and 3A, biopsy probe assembly 14 includes a probe housing 36, a vacuum cannula 38, a stylet cannula 40, a cutter cannula 42, a sample manifold 48, and a sample container 50. The portion of vacuum cannula 38, stylet cannula 40, cutter cannula 42 that extends distally on longitudinal axis 44 from front plate 36-2 of biopsy probe assembly 14 of biopsy apparatus 10 is referred to herein as a biopsy needle 52.

Probe housing 36 is formed as an L-shaped structure having an elongate portion 36-1 and a front plate 36-2. When biopsy probe assembly 14 is attached to driver assembly 12, front plate 36-2 is positioned distally adjacent to an entirety of front surface 16-1 of driver housing 16, i.e., so as to shield the entirety of front surface 16-1 of the non-disposable driver assembly from contact with a patient.

Vacuum cannula 38, stylet cannula 40, and cutter cannula 42 are coaxially arranged along a longitudinal axis 44 in a nested tube arrangement, with vacuum cannula 38 being the innermost tube, cutter cannula 42 being the outermost tube, and stylet cannula 40 being the intermediate tube that is interposed between vacuum cannula 38 and cutter cannula 42. In other words, vacuum cannula 38 is positioned inside stylet cannula 40, and stylet cannula 40 is positioned inside cutter cannula 42.

Vacuum cannula 38 is coupled in fluid communication with vacuum source 22 via sample manifold 48.

Stylet cannula 40 includes a sample notch 40-1 and a piercing tip 40-2. Sample notch 40-1 is formed as an elongate opening in stylet cannula 40 to facilitate a reception of tissue into a lumen of stylet cannula 40. Cutter cannula 42 has a distal cutting end 42-1.

Stylet cannula 40 and cutter cannula 42 are jointly connected to piercing shot drive 34-2. Upon a first actuation of prime/pierce button 28-2, stylet cannula 40 and cutter cannula 42 are translated in unison in proximal direction 46-1 along longitudinal axis 44 to position piercing shot drive 34-2, stylet cannula 40, and cutter cannula 42 in a ready, i.e., cocked position. A second actuation of prime/pierce button 28-2 effects a piercing shot, wherein piercing shot drive 34-2 is released from the cocked position, and in turn rapidly propels stylet cannula 40 and cutter cannula 42 in distal direction 46-2 along longitudinal axis 44 toward a distal-most position of the combined elements, e.g., within the patient.

Cutter cannula 42 is connected to cutter drive 30-2, and is individually retracted or extended along longitudinal axis 44 by activation of cutter module 30 of biopsy probe assembly 14 by activation of sample button 28-1 of user interface 28 to initiate a sample sequence. For example, cutter cannula 42 may be translated and retracted axially along longitudinal axis 44 to expose sample notch 40-1 during a sample sequence so that tissue may be pulled by vacuum into the lumen of stylet cannula 40 by vacuum provided by vacuum cannula 38. Thereafter, cutter cannula 42 may have a rotational cutting motion and may be translated axially along longitudinal axis 44 to extend over sample notch 40-1 such that distal cutting end 42-1 of cutter cannula 42 severs the tissue that is pulled by vacuum into the lumen of stylet cannula 40 by vacuum provided by vacuum cannula 38.

Stylet cannula 40 is individually retracted or extended along longitudinal axis 44 by activation of transport module 32 of biopsy probe assembly 14, so as to aid in delivery of a tissue sample into the lumen of vacuum cannula 38. Vacuum cannula 38 then transports the tissue sample, via vacuum, to sample manifold 48 by the vacuum supplied to sample manifold 48 by vacuum source 22.

Referring to FIGS. 3A-3C, sample manifold 48 is configured as an L-shaped structure having a vacuum chamber portion 48-1 and a collection chamber portion 48-2. Vacuum chamber portion 48-1 includes a vacuum input port 48-3 that is arranged to sealably engage vacuum source port 22-3 (see FIG. 2) of vacuum source 22 of driver assembly 12 when biopsy probe assembly 14 is attached to driver assembly 12. Blotting papers may be placed in vacuum chamber portion 48-1 in a region between vacuum input port 48-3 and collection chamber portion 48-2.

Vacuum chamber portion 48-1 is connected in fluid communication with collection chamber portion 48-2. A proximal end portion 38-1 of vacuum cannula 38 passes into vacuum chamber portion 48-1 and is in fluid communication with collection chamber portion 48-2.

Referring to FIGS. 3B and 3C, collection chamber portion 48-2 of sample manifold 48 defines a receptacle 48-4 that is sized and arranged to removably receive, and mount, sample container 50. Referring also to FIGS. 1, 2, and 3A, when sample container 50 is mounted in receptacle 48-4 of collection chamber portion 48-2 of sample manifold 48, sample container 50 is interposed between vacuum source 22 and proximal end portion 38-1 of vacuum cannula 38, such that sample container 50 is in direct fluid communication with proximal end portion 38-1 of vacuum cannula 38, and sample container 50 also is in direct fluid communication with vacuum input port 48-3 of vacuum chamber portion 48-1.

Thus, vacuum generated by vacuum source 22 is delivered to proximal end portion 38-1 of vacuum cannula 38 via sample manifold 48 and sample container 50. Accordingly, when vacuum is applied by vacuum source 22 at vacuum input port 48-3 of vacuum chamber portion 48-1 of sample manifold 48, the vacuum passes through sample container 50, such that a tissue sample severed by cutter cannula 42 at sample notch 40-1 of stylet cannula 40 may be transported by vacuum, through vacuum cannula 38, and into sample container 50.

Referring to FIGS. 3B and 3C, receptacle 48-4 of collection chamber portion 48-2 of sample manifold 48 has an open end 48-5, an interior side wall 48-6, and a mounting pin 48-7. Collection chamber portion 48-2 has an insertion axis 48-8. Mounting pin 48-7 is formed on interior side wall 48-6 of receptacle 48-4 and projects inwardly from the interior side wall 48-6 toward insertion axis 48-8.

In the present embodiment, sample container 50 is configured so as to mate with receptacle 48-4 of collection chamber portion 48-2 of sample manifold 48 in such a way that sample container 50 can only be loaded into receptacle 48-4 in one orientation and in one direction, thereby reducing operator errors after removal and reloading sample container 50 to take additional tissue samples.

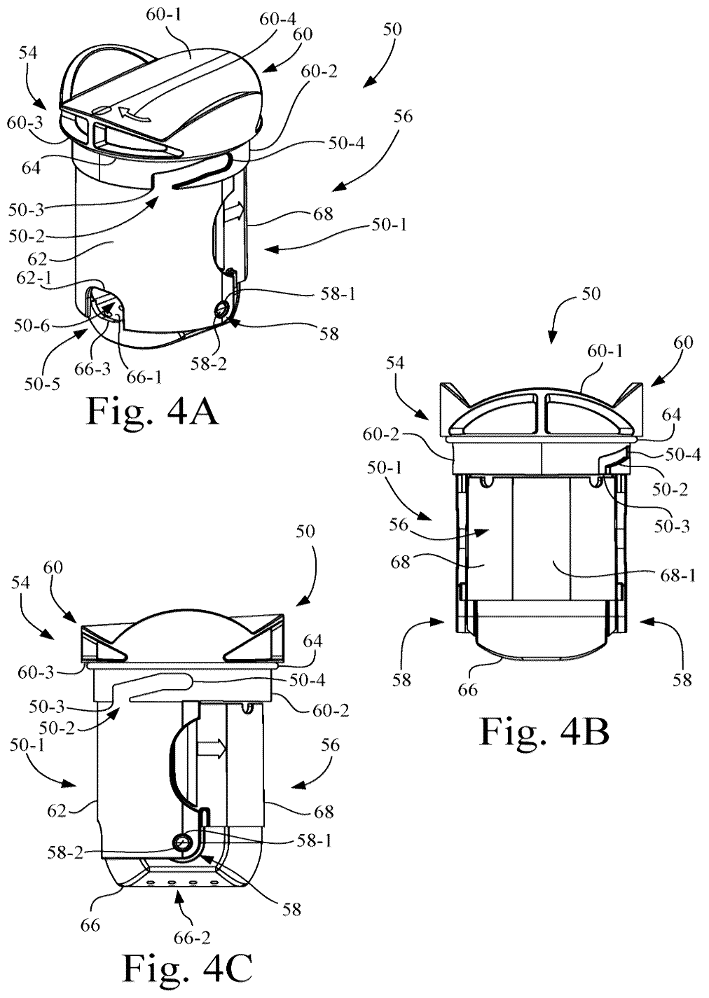

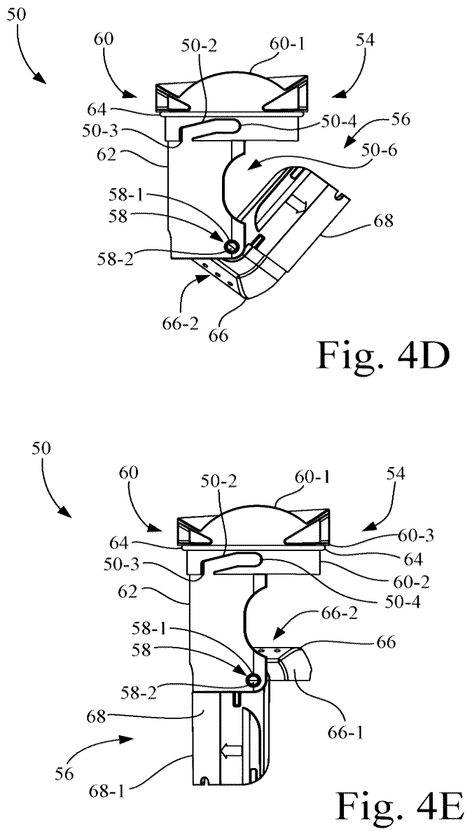

Referring also to FIGS. 4A-4E, sample container 50 has a cap portion 54 and a basket portion 56, which are joined by a hinge 58, and which when in a closed position, fit together to define a cylindrical side wall 50-1. Each of cap portion 54 and a basket portion 56 may be made of a rigid plastic, and may have smooth surface features to reduce radiograph visibility impact if tissue sample radiographs are desired, or required. The rigid plastic may also be selected for compatibility with formalin.

Referring also to FIGS. 3B and 3C, sample container 50 is sized and configured to be removably received in receptacle 48-4 of collection chamber portion 48-2 of sample manifold 48. For example, sample container 50 and interior side wall 48-6 of receptacle 48-4 may have complementary cylindrical shapes and are axially aligned along an insertion axis 48-8. Cylindrical side wall 50-1 of sample container 50 has an outside diameter that is selected to be slidably received by cylindrical interior side wall 48-6 of receptacle 48-4. As used herein, the term “cylindrical” means a generally arcuate-shaped annular contour that may include flats, ledges, and/or other surface features not associated with a pure cylinder.

Referring to FIGS. 4A-4E, sample container 50 includes a mounting channel 50-2 that is sized and positioned to engage and follow mounting pin 48-7 of receptacle 48-4 of sample manifold 48 (see FIG. 3C). Mounting channel 50-2 spirals an arcuate distance of less than one turn of sample container 50, e.g., one-eighth turn to one-quarter of the circumference of sample container 50 (depending on design parameters), downwardly from cap portion 54 along cylindrical side wall 50-1. Mounting channel 50-2 has an open end 50-3 and a closed end 50-4. Open end 50-3 is positioned to receive mounting pin 48-7 of receptacle 48-4 of collection chamber portion 48-2 of sample manifold 48 and to provide an initial indexing (rotational orientation) of sample container 50 relative to receptacle 48-4 of collection chamber portion 48-2 of sample manifold 48 about insertion axis 48-8.

When mounting pin 48-7 of receptacle 48-4 of collection chamber portion 48-2 of sample manifold 48 is received in open end 50-3 of mounting channel 50-2 of sample container 50, a rotation (clockwise in the present example) of sample container 50 causes mounting pin 48-7 to follow the spiral shape of mounting channel 50-2 toward closed end 50-4 as sample container 50 is rotated, so as to pull sample container 50 along insertion axis 48-8 into receptacle 48-4 of collection chamber portion 48-2 of sample manifold 48.

It is contemplated that mounting channel 50-2 could include multiple spiral channels formed in sample container 50, if desired. In this case, receptacle 48-4 of collection chamber portion 48-2 of sample manifold 48 may have a number of mounting pins that correspond to the number of spiral channels formed in sample container 50, each of which being received in a respective spiral channel of sample container 50 as sample container 50 is received in receptacle 48-4 of collection chamber portion 48-2 of sample manifold 48.

Referring to FIGS. 4A-4E, cap portion 54 of sample container 50 includes a lid 60 and a partial side wall 62 that may be formed as a one-piece unitary structure. Lid 60 includes a domed cap 60-1, an annular rim 60-2, and an annular lip 60-3. Annular rim 60-2 joins domed cap 60-1 to define annular lip 60-3. Mounting channel 50-2 is formed in, and spirals around, annular rim 60-2. Domed cap 60-1 may include an arrow indicia 60-4, e.g., raised or embossed, to indicate a rotation direction for effecting the mounting of sample container 50 in receptacle 48-4 of collection chamber portion 48-2 of sample manifold 48.

A seal 64, such as an O-ring, may be placed around annular rim 60-2, such that when sample container 50 is mounted in receptacle 48-4 of collection chamber portion 48-2 of sample manifold 48, seal 64 provides a vacuum-tight seal between sample container 50 and receptacle 48-4 of collection chamber portion 48-2 of sample manifold 48. Advantageously, a reverse rotation of sample container 50 causes mounting channel 50-2 to follow the spiral shape of mounting pin 48-7 toward open end 50-3, so as to pull sample container 50 along insertion axis 48-8 out of receptacle 48-4 to aid in releasing the vacuum seal.

In one embodiment, at least the domed cap 60-1 of cap portion 54 is transparent, or translucent, so as to permit visual inspection of the contents of sample container 50 without having to remove sample container 50 from receptacle 48-4 of collection chamber portion 48-2 of sample manifold 48. By rounding domed cap 60-1 of cap portion 54, the interior of sample container 50 may be observed from multiple angles while holding biopsy apparatus 10 or observing the biopsy procedure. Also, domed cap 60-1 of cap portion 54 may include a magnification feature to further improve visibility of the contents of sample container 50, e.g., during a biopsy.

Referring to FIGS. 4C-4E, basket portion 56 includes a floor 66 and a partial side wall 68 that may be formed as a one-piece unitary structure. Partial side wall 68 extends upwardly from floor 66. In the present embodiment, floor 66 has a concave interior surface 66-1 and includes a plurality of holes 66-2 (see also FIG. 4A). The plurality of holes 66-2 in floor 66 of sample container 50 allow vacuum to travel through sample container 50 during a biopsy procedure, and the tissue specimens will be delivered from biopsy needle 52 onto concave interior surface 66-1 of floor 66 of sample container 50. In addition, following the biopsy procedure, sample container 50 may be placed directly into a specimen jar containing formalin, wherein the plurality of holes 66-2 in floor 66 of sample container 50 allow for formalin to infuse into sample container 50.

Referring to FIGS. 4A-4E, cap portion 54 and a basket portion 56 are joined by hinge 58 located at a bottom portion of sample container 50. Hinge 58 has engageable hinge features formed on both of cap portion 54 and a basket portion 56. In the present embodiment, for example, hinge 58 includes a pair of opposed apertures 58-1 formed in a lower distal portion of partial side wall 62 of cap portion 54 and a corresponding pair of opposed pins 58-2 that extend radially outwardly from partial side wall 68 of basket portion 56 just above floor 66, wherein the pair of opposed pins 58-2 are received in the pair of opposed apertures 58-1, e.g., holes, to form a pivot joint. This pivot joint provides case of access to the tissue specimens contained in sample container 50. The hinge action of cap portion 54 and basket portion 56 also may create a scooping action to aid in specimen retrieval as sample container 50 is opened.

It is also contemplated that the engageable hinge features of the pin/hole arrangement can be reversed, if desired, such that the pins are on partial side wall 62 of cap portion 54 and the holes are in partial side wall 68 of the basket portion 56.

As an alternative to forming apertures 58-1 as holes, it is contemplated that apertures 58-1 may be formed as slots or channels, such that the pair of opposed pins 58-2 of hinge 58 may be readily disengaged from apertures 58-1 when sample container 50 is opened, so as to facilitate easy removal of basket portion 56 from cap portion 54 after basket portion 56 has been opened to a predefined position relative to cap portion 54, if desired.

FIGS. 4A-4C depict sample container 50 in a closed position, with partial side wall 62 of cap portion 54 overlapping partial side wall 68 of basket portion 56 to form a snap fit. Advantageously, by joining cap portion 54 and basket portion 56 with hinge 58, an operator must take a deliberate action to open sample container 50 to remove the tissue samples, and there is no risk of accidentally detaching a lid or piece of the container when removing it from biopsy apparatus 10. The tissue samples (specimens) are retained in sample container 50 until opened, reducing the risk of tissue sample loss during transport.

Sample container 50 must be in the closed position depicted in FIGS. 4A-4C in order for sample container 50 to be receivable into receptacle 48-4 of collection chamber portion 48-2 of sample manifold 48 (see also FIGS. 3B and 3C) so that sample container 50 can be seated in and mounted to sample manifold 48 (see also FIG. 3A). As shown in FIGS. 3B and 4A, when sample container 50 is in the closed position, cap portion 54 and basket portion 56 together define an aperture 50-5 and an interior sample chamber 50-6. Proximal end portion 38-1 of vacuum cannula 38 extends to aperture 50-5 to deliver, via vacuum, tissue samples from biopsy needle 52 into interior sample chamber 50-6 and onto concave interior surface 66-1 of floor 66 having the plurality of holes 66-2 (see also FIG. 4C). In the present embodiment, aperture 50-5 of sample container 50 is formed by locating a semicircular aperture 62-1 in partial side wall 62 of cap portion 54 adjacent to a semicircular aperture 66-3 located above floor 66 of basket portion 56, when sample container 50 is in the closed position.

FIGS. 4D and 4E show a sequence of opening sample container 50, wherein one or both of cap portion 54 and basket portion 56 are pivoted around hinge 58 such that the contents of sample container 50 may be accessed. FIG. 4D shows sample container 50 at an intermediate open position, and FIG. 4E shows sample container 50 is a completely open position. When sample container 50 is placed in the completely open position and oriented on its side, a flat exterior feature 68-1 on partial side wall 68 of basket portion 56 allows sample container 50 to rest on a flat surface, such as a table top or procedure tray, thereby reducing the risk of sample container 50 rolling off the flat surface.

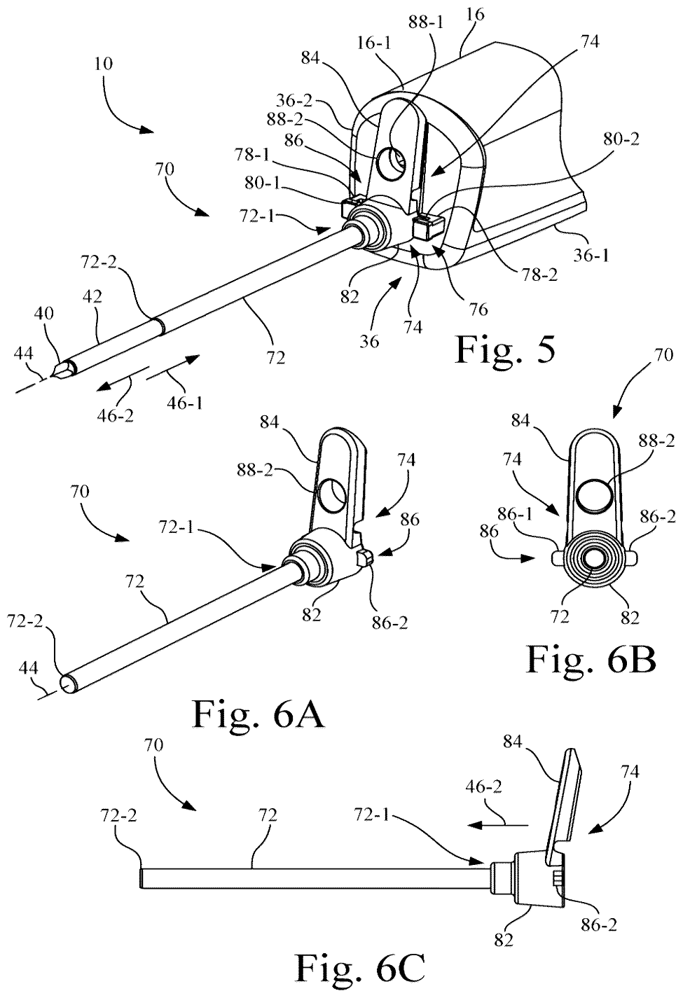

Referring to FIG. 5, with reference to FIG. 1, in some procedures an operator may desire to use a coaxial introducer cannula 70 to maintain an access path to the biopsy site, e.g., to the lesion, such that biopsy needle 52 of biopsy apparatus 10 may be removed from the biopsy site, while maintaining the ability to reinsert biopsy needle 52, or to insert another medical apparatus, such as a tissue marker deploying device, at the biopsy site.

Referring also to FIGS. 6A-6C, coaxial introducer cannula 70 includes a coaxial cannula 72 and a hub 74. Coaxial cannula 72 may be formed as an elongate tube, e.g., a metal tube such as a stainless steel tube, having a proximal portion 72-1 and a distal end 72-2. Hub 74 is made from a rigid plastic which is fixedly attached (e.g., overmolded, adhesively connected, etc.) to a proximal portion 72-1 of coaxial cannula 72.

Coaxial cannula 72 of coaxial introducer cannula 70 is sized to be coaxially and slidably received along longitudinal axis 44 over biopsy needle 52 formed by stylet cannula 40 and cutter cannula 42. Hub 74 of coaxial introducer cannula 70 is configured for releasable attachment to front plate 36-2 of probe housing 36 of biopsy apparatus 10.

In the present embodiment, referring also to FIG. 5, front plate 36-2 of probe housing 36 includes a catch 76 formed as a set of slotted protrusions 78-1, 78-2 that protrude outwardly from front plate 36-2 of probe housing 36 of biopsy apparatus 10. Slotted protrusions 78-1, 78-2 have respective opposed slots 80-1, 80-2 which face in a direction toward longitudinal axis 44.

Referring to FIGS. 5 and 6A-6C, in the present embodiment hub 74 includes a hub body 82, latching lever 84, and a latch 86. Latching lever 84 is an elongate arm that extends radially from hub body 82 relative to longitudinal axis 44. Latch 86 is configured to rotatably engage catch 76 of front plate 36-2 of biopsy probe assembly 14 of biopsy apparatus 10. In the present embodiment, latch 86 is in the form of a set of tabs 86-1, 86-2 that extends radially outwardly from hub body 82.

As shown in FIG. 5, latching lever 84 is longer than a height of front plate 36-2 of probe housing 36 and driver assembly 12, so that latching lever 84 can be reached and rotationally operated by the operator's thumb or finger while the operator grasps biopsy apparatus 10 with the same hand, thereby facilitating single-handed rotation of the coaxial introducer cannula relative to front plate 36-2 of biopsy probe assembly 14 of biopsy apparatus 10, so as to effect a respective engagement and disengagement of catch 76 of coaxial introducer cannula 70 and latch 86 of front plate 36-2 of biopsy probe assembly 14 of biopsy apparatus 10.

Also, optionally, referring to FIGS. 5, 6A and 6B, a first lock feature 88-1 may be provided at front plate 36-2 of probe housing 36 and a second lock feature 88-2 may be provided on hub 74 of coaxial introducer cannula 70, which when engaged, resists rotation of coaxial introducer cannula 70 about longitudinal axis 44, i.e., relative to biopsy apparatus 10. More particularly, second lock feature 88-2 may be positioned on or in latching lever 84.

Referring to FIGS. 5 and 6A-6C, during installation of coaxial introducer cannula 70 on biopsy apparatus 10, coaxial cannula 72 of coaxial introducer cannula 70 is coaxially and slidably received along longitudinal axis 44 over biopsy needle 52 formed by stylet cannula 40 and cutter cannula 42. With the set of tabs 86-1, 86-2 of hub 74 of coaxial introducer cannula 70 rotationally misaligned from slotted protrusions 78-1, 78-2 of front plate 36-2 of probe housing 36 shown in FIG. 5, coaxial introducer cannula 70 is moved axially along longitudinal axis 44 until hub 74 is axially seated against front plate 36-2 of probe housing 36. As shown in FIG. 6C, latching lever 84 is angled proximally relative to hub body 82, such that latching lever 84 engages front plate 36-2 shown in FIG. 5 prior to being axially seated against front plate 36-2, and latching lever 84 is deflected, as a cantilever spring, in distal direction 46-2.

Latching lever 84 of hub 74 is then rotated about longitudinal axis 44 to a latched position, depicted in FIG. 5, wherein latch 86, e.g., the set of tabs 86-1, 86-2, of hub 74 is received in the catch 76, e.g., the opposed slots 80-1, 80-2 of the set of slotted protrusions 78-1, 78-2, of front plate 36-2 of probe housing 36, so as to connect coaxial introducer cannula 70 to biopsy apparatus 10 to thereby prevent axial movement of coaxial introducer cannula 70 relative to biopsy apparatus 10 along longitudinal axis 44. The rotational motion of latching lever 84 may be either clockwise or counterclockwise to allow for ambidextrous operation. First lock feature 88-1 of front plate 36-2 of probe housing 36 is now also engaged with the second lock feature 88-2 on latching lever 84 of hub 74 of coaxial introducer cannula 70, so as to resist rotation of coaxial introducer cannula 70 about longitudinal axis 44.

In the present embodiment, first lock feature 88-1 of front plate 36-2 of probe housing 36 and second lock feature 88-2 of hub 74 of coaxial introducer cannula 70 are complementary engagement features, such as a hole/protrusion arrangement or a magnetic arrangement, which when engaged will resist, but not prohibit, rotation of coaxial introducer cannula 70 about longitudinal axis 44. For example, second lock feature 88-2 may be a notch or opening formed in the proximal surface of latching lever 84 of hub 74, and first lock feature 88-1 of front plate 36-2 of probe housing 36 may be a raised distally facing area, e.g., a pin and/or headlamp, on front plate 36-2 that mates with the notch formed in latching lever 84, or vice-versa, or both, so as to provide resistance to a rotation of coaxial introducer cannula 70 about longitudinal axis 44, i.e., relative to biopsy apparatus 10.

In order to disconnect coaxial introducer cannula 70 from biopsy apparatus 10, latching lever 84 is rotated about longitudinal axis 44 to disengage latch 86, e.g., the set of tabs 86-1, 86-2, of hub 74 of coaxial introducer cannula 70 from catch 76, e.g., the opposed slots 80-1, 80-2 of the set of slotted protrusions 78-1, 78-2, of front plate 36-2 of probe housing 36. The rotational motion of latching lever 84 may be either clockwise or counterclockwise to allow for ambidextrous operation. Coaxial introducer cannula 70 is now free to move axially along longitudinal axis 44 in distal direction 46-2 away from front plate 36-2 of probe housing 36 to remove coaxial introducer cannula 70 from biopsy needle 52 of biopsy apparatus 10. Since latching lever 84 is resilient, as latching lever 84 returns to its pre-deflection position, and the cantilever spring action generated by latching lever 84 pushes against front plate 36-2 so as to aid in moving coaxial introducer cannula 70 in distal direction 46-2 away from its seated position.

Alternatively, in one magnet configuration for first lock feature 88-1 and second lock feature 88-2, for example, front plate 36-2 of probe housing 36 may have a center magnet having a polarity to attract a magnet embedded in, or attached to, latching lever 84 of hub 74 when coaxial introducer cannula 70 is in the latched position, so as to resist rotation of coaxial introducer cannula 70 relative to biopsy apparatus 10. Front plate 36-2 of probe housing 36 also may have two rotationally spaced magnets, one on either side of the center magnet, having a polarity the same as that of the hub magnet, so as to repel the hub magnet in distal direction 46-2 to assist the operator in axial removal of coaxial introducer cannula 70 from biopsy probe assembly 14 of biopsy apparatus 10 after latching lever 84 has been rotated to disconnect coaxial introducer cannula 70 from biopsy apparatus 10.

It is contemplated that coaxial introducer cannula 70 may be used with, and connected to, other types of biopsy apparatus, such as a trocar adapted to include catch 76, e.g., the slotted protrusions 78-1, 78-2, and to optionally include first lock feature 88-1.

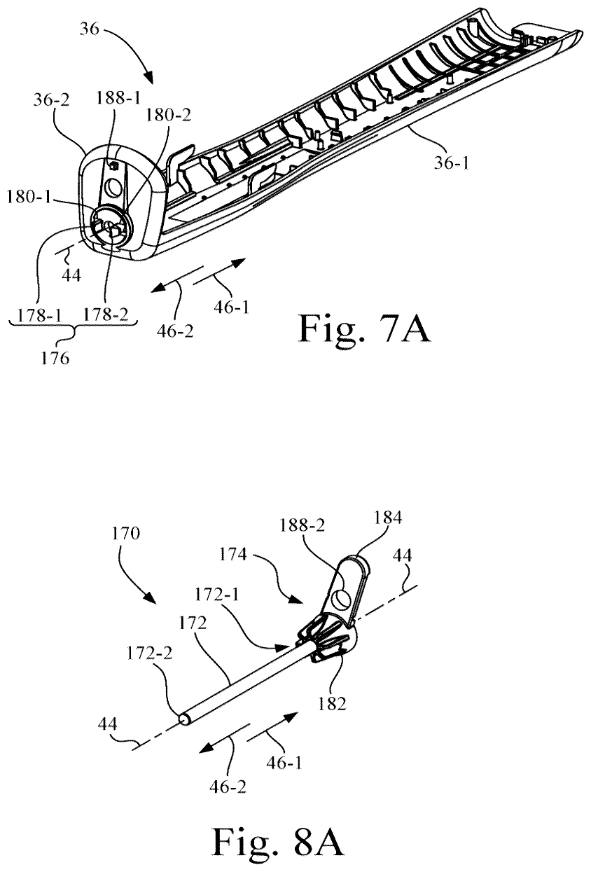

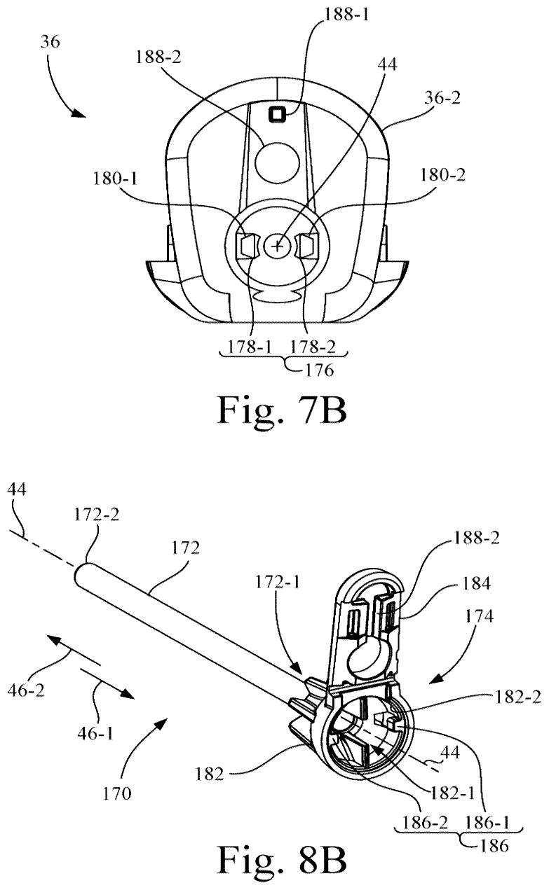

FIGS. 7A, 7B, 8A, and 8B are directed to another embodiment for connection of a coaxial introducer cannula in accordance with the present invention, with probe housing 36.

FIGS. 7A, 7B, 8A, and 8B are directed to another embodiment for connection of a coaxial introducer cannula with probe housing 36 of biopsy apparatus 10.

Referring to FIGS. 8A and 8B, in the present embodiment, a coaxial introducer cannula 170 includes a coaxial cannula 172 and a hub 174. Coaxial cannula 172 may be formed as an elongate tube, e.g., a metal tube such as a stainless steel tube, having a proximal portion 172-1 and a distal end 172-2. Hub 174 is made from a rigid plastic which is fixedly attached (e.g., overmolded, adhesively connected, etc.) to a proximal portion 172-1 of coaxial cannula 172.

Coaxial cannula 172 of coaxial introducer cannula 170 is sized to be coaxially and slidably received along longitudinal axis 44 over biopsy needle 52 formed by stylet cannula 40 and cutter cannula 42 (see also FIG. 1). Hub 174 of coaxial introducer cannula 170 is configured for releasable attachment to front plate 36-2 of probe housing 36 of biopsy apparatus 10 (see FIGS. 7A and 7B).

As shown in FIGS. 7A and 7B, in the present embodiment, front plate 36-2 of probe housing 36 includes a catch 176 formed as a set of slotted protrusions 178-1, 178-2 that protrude outwardly in distal direction 46-2 from front plate 36-2 of probe housing 36 of biopsy apparatus 10. Slotted protrusions 178-1, 178-2 have respective opposed slots 180-1, 180-2 which face in a direction away from longitudinal axis 44.

As shown in FIGS. 8A and 8B, in the present embodiment, hub 174 includes a hub body 182, latching lever 184, and a latch 186. Latch 186 is configured to rotatably engage catch 176 of front plate 36-2 of biopsy probe assembly 14 of biopsy apparatus 10. Latching lever 184 is an elongate arm that extends radially from hub body 182 relative to longitudinal axis 44. As shown in FIG. 8B, hub body 182 has a cylindrical recess 182-1 defining a side wall 182-2. Latch 186 is in the form of a set of tabs 186-1, 186-2 that extend radially inwardly from side wall 182-2 of cylindrical recess 182-1 of hub body 182, toward longitudinal axis 44.

Latching lever 184 is longer than a height of front plate 36-2 of probe housing 36 and driver assembly 12 (see FIG. 1), so that latching lever 184 can be reached and rotationally operated by the operator's thumb or finger while the operator grasps biopsy apparatus 10 with the same hand, thereby facilitating single-handed rotation of coaxial introducer cannula 170 relative to front plate 36-2 of biopsy probe assembly 14 of biopsy apparatus 10, so as to effect a respective engagement or disengagement of latch 186 of coaxial introducer cannula 170 with catch 176 of front plate 36-2 of biopsy probe assembly 14 of biopsy apparatus 10.

Also, optionally, a first lock feature 188-1 may be provided at front plate 36-2 of probe housing 36 and a second lock feature 188-2 may be provided on hub 174 of coaxial introducer cannula 170, which when engaged, resists rotation of coaxial introducer cannula 170 about longitudinal axis 44, i.e., relative to biopsy apparatus 10. More particularly, second lock feature 188-2 may be positioned on or in latching lever 184.

Referring to FIGS. 7A, 7B, 8A, and 8B (with reference to FIG. 1), during installation of coaxial introducer cannula 170 on biopsy apparatus 10, coaxial cannula 172 of coaxial introducer cannula 170 is coaxially and slidably received along longitudinal axis 44 over biopsy needle 52 formed by stylet cannula 40 and cutter cannula 42 (see FIG. 1). With the latch 186, e.g., the set of tabs 186-1, 186-2, of hub 174 of coaxial introducer cannula 170 rotationally misaligned from catch 176, e.g., the slotted protrusions 178-1, 178-2, of front plate 36-2 of probe housing 36, coaxial introducer cannula 170 is moved axially along longitudinal axis 44 until hub 174 and is axially seated against front plate 36-2 of probe housing 36. Latching lever 184 may be angled proximally relative to hub body 182, such that latching lever 184 engages front plate 36-2 prior to being axially seated against front plate 36-2 and latching lever 184 is deflected, as a cantilever spring, in distal direction 46-2.

Latching lever 184 of hub 174 is then rotated about longitudinal axis 44 to a latched position, wherein latch 186, e.g., the set of tabs 186-1, 186-2 of hub 174 are respectively received in catch 176, e.g., the opposed slots 180-1, 180-2 of the set of slotted protrusions 178-1, 178-2, of front plate 36-2 of probe housing 36, so as to connect coaxial introducer cannula 170 to probe housing 36, and thus to biopsy apparatus 10, to thereby prevent axial movement of coaxial introducer cannula 170 relative to biopsy apparatus 10 along longitudinal axis 44. The rotational motion of latching lever 184 may be either clockwise or counterclockwise to allow for ambidextrous operation. First lock feature 188-1 of front plate 36-2 of probe housing 36 is now also engaged with the second lock feature 188-2 on latching lever 184 of hub 174 of coaxial introducer cannula 170, so as to resist rotation of coaxial introducer cannula 170 about longitudinal axis 44.

In the present embodiment, first lock feature 188-1 of front plate 36-2 of probe housing 36 and second lock feature 188-2 of hub 174 of coaxial introducer cannula 170 are complementary engagement features, such as a slot/protrusion arrangement, which when engaged will resist, but not prohibit, rotation of coaxial introducer cannula 170 about longitudinal axis 44. For example, second lock feature 188-2 may be an opening, e.g., hole and/or slot, formed in a proximal surface of latching lever 184 of hub 174, and first lock feature 188-1 of front plate 36-2 of probe housing 36 may be a raised distally facing area, e.g., a pin and/or headlamp, on front plate 36-2 that mates with the opening formed in latching lever 184, or vice-versa, or both, so as to provide resistance to a rotation of coaxial introducer cannula 170 about longitudinal axis 44, i.e., relative to biopsy apparatus 10.

In order to disconnect coaxial introducer cannula 170 from biopsy apparatus 10, latching lever 184 is rotated about longitudinal axis 44 to disengage latch 186, e.g., the set of tabs 186-1, 186-2, of hub 174 of coaxial introducer cannula 170 from catch 176, e.g., the opposed slots 180-1, 180-2 of the set of slotted protrusions 178-1, 178-2, of front plate 36-2 of probe housing 36. The rotational motion of latching lever 184 may be either clockwise or counterclockwise to allow for ambidextrous operation. Coaxial introducer cannula 170 is now free to move axially along longitudinal axis 44 in distal direction 46-2 away from front plate 36-2 of probe housing 36 to remove coaxial introducer cannula 170 from biopsy needle 52 of biopsy apparatus 10.

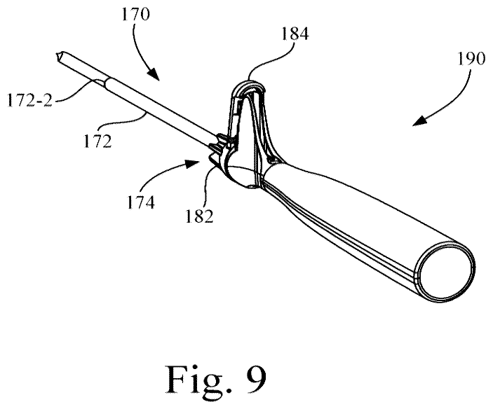

Referring to FIG. 9, it is contemplated that coaxial introducer cannula 170 may be used with, and connected to, other types of biopsy apparatus, such as a trocar 190 adapted to include catch 176, e.g., the slotted protrusions 178-1, 178-2, and to optionally include first lock feature 188-1.

The following items also relate to the invention:

In one form, the invention relates to a biopsy apparatus, or alternatively to a biopsy probe assembly, that includes a biopsy needle, a sample manifold, and a sample container. The sample manifold is coupled to the biopsy needle. The sample manifold has a receptacle and an insertion axis. The sample receptacle has an interior side wall and a mounting pin that projects inwardly from the interior side wall toward the insertion axis. The sample container is configured for insertion into the receptacle. The sample container includes a mounting channel that is sized and positioned (structured) to engage and follow the mounting pin of the receptacle as the sample container is rotated.

Optionally, the mounting channel is configured to spiral an arcuate distance of less than one turn of the sample container.

Optionally, the mounting channel has an open end and a closed end. The open end is positioned to receive the mounting pin of the sample manifold to provide a rotational orientation of the sample container relative to the receptacle of the sample manifold about the insertion axis.

The biopsy apparatus may be configured such that, when the mounting pin of the receptacle is received in the open end of the mounting channel of the sample container, a rotation of the sample container causes the mounting pin to follow the spiral shape of the mounting channel toward the closed end, so as to pull the sample container along the insertion axis into the receptacle of the manifold.

The sample container may have a cap portion and a basket portion that are joined by a hinge to facilitate a pivot of one or both of the cap portion and the basket portion around the hinge between a closed position and an open position. The open position provides access to contents of the sample container.

The biopsy apparatus may be configured such that, when the sample container is in the closed position, a partial side wall of the cap portion overlaps a partial side wall of the basket portion to form a snap fit.

The cap portion of the sample container may include a lid and a partial side wall. The lid may include a domed cap and an annular rim. The annular rim may join the domed cap to define an annular lip. The mounting channel may be formed in, and spiral around, the annular rim.

The domed cap may be transparent or translucent to permit visual inspection of the contents of the sample container without having to remove the sample container from the receptacle of the manifold.

The domed cap may include a magnification feature.

A seal may be placed around the annular rim to provide a vacuum-tight seal between the sample container and the receptacle of the manifold.

The basket portion may include a floor having a concave interior surface and a plurality of holes. The biopsy apparatus may be configured such that the plurality of holes allows a vacuum to travel through the sample container to deliver a tissue sample from the biopsy needle onto the concave interior surface of the floor.

The cap portion and the basket portion of the sample container together may define an aperture and an interior sample chamber. The biopsy needle may include a vacuum cannula having a proximal end portion that extends to the aperture to deliver, via vacuum, tissue samples from the biopsy needle into the interior sample chamber.

The aperture of the sample container may be formed by a first semicircular aperture of the cap portion positioned adjacent a second semicircular aperture of the basket portion when the sample container is in a closed position.

Any of the features of items [00102] to [00114] may be combined in a biopsy apparatus, or alternatively in a biopsy probe assembly.

Optionally, a coaxial introducer cannula for use with the biopsy apparatus may be provided. The biopsy apparatus has a front plate having a catch, and a biopsy needle extends from the front plate on a longitudinal axis. The coaxial introducer cannula includes a coaxial cannula and a hub. The coaxial cannula is sized to be coaxially and slidably received over the biopsy needle. The hub is fixedly attached to a proximal portion of the coaxial cannula. The hub has a hub body, a latching lever, and a latch. The latch is configured to rotatably engage the catch. The latching lever extends radially from the hub body relative to the longitudinal axis. The latching lever is longer than a height of the front plate so that the latching lever can be reached and rotationally operated to rotate the hub relative to the front plate of the biopsy apparatus, thereby facilitating single-handed rotation of the coaxial introducer cannula relative to the front plate, so as to effect a respective engagement or disengagement of the latch of the coaxial introducer cannula with the catch of the front plate.

A rotational motion of the latching lever may be either clockwise or counterclockwise to allow for ambidextrous operation.

The latching lever may be angled proximally relative to the hub body, such that the latching lever engages the front plate prior to the hub being axially seated against the front plate, such that the latching lever is deflected in a distal direction.

A first lock feature may be on the front plate and a second lock feature may be on the hub, which when engaged will resist, but not prohibit, rotation of the coaxial introducer cannula about the longitudinal axis.

The second lock feature may be positioned on or in the latching lever.

The first lock feature and the second lock feature may form a hole/protrusion arrangement or a magnetic arrangement.

The catch may be a set of slotted protrusions that protrude outwardly in a distal direction from the front plate. The slotted protrusions may have respective opposed slots which face in a direction away from the longitudinal axis. The hub body may have a cylindrical recess defining a side wall, wherein the latch is in the form of a set of tabs that extend radially inwardly toward the longitudinal axis from the side wall of the cylindrical recess of the hub body.

As used herein, “generally,” and other words of degree are relative modifiers intended to indicate permissible variation from the characteristic so modified. It is not intended to be limited to the absolute value or characteristic which it modifies but rather possessing more of the physical or functional characteristic than its opposite, and approaching or approximating such a physical or functional characteristic.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A coaxial introducer cannula for use with a biopsy apparatus, the biopsy apparatus having a front plate having a catch, and a biopsy needle that extends from the front plate on a longitudinal axis, the coaxial introducer cannula comprising:
a coaxial cannula sized to be coaxially and slidably received over the biopsy needle; and
a hub, the hub being fixedly attached to a proximal portion of the coaxial cannula, the hub having a hub body, a latching lever, and a latch, the latch configured to rotatably engage the catch, and wherein the latching lever extends radially from the hub body relative to the longitudinal axis, the latching lever being longer than a height of the front plate so that the latching lever can be reached and rotationally operated to rotate the hub relative to the front plate of the biopsy apparatus, thereby facilitating single-handed rotation of the coaxial introducer cannula relative to the front plate, so as to effect a respective engagement or disengagement of the latch of the coaxial introducer cannula with the catch of the front plate.

2. The coaxial introducer cannula of claim 1, wherein a rotational motion of the latching lever may be either clockwise or counterclockwise to allow for ambidextrous operation.

3. The coaxial introducer cannula of claim 1, wherein the latching lever is angled proximally relative to the hub body, such that the latching lever engages the front plate prior to the hub being axially seated against the front plate, such that the latching lever is deflected in a distal direction.

4. The coaxial introducer cannula of claim 1, comprising a first lock feature on the front plate and a second lock feature on the hub, which when engaged will resist, but not prohibit, rotation of the coaxial introducer cannula about the longitudinal axis.

5. The coaxial introducer cannula of claim 4, wherein the second lock feature is positioned on or in the latching lever.

6. The coaxial introducer cannula of claim 4, wherein the first lock feature and the second lock feature form a hole/protrusion arrangement or a magnetic arrangement.

7. The coaxial introducer cannula of claim 1, wherein:
the catch is a set of slotted protrusions that protrude outwardly in a distal direction from the front plate, the slotted protrusions having respective opposed slots which face in a direction away from the longitudinal axis; and
the hub body having a cylindrical recess defining a side wall, wherein the latch is in the form of a set of tabs that extend radially inwardly toward the longitudinal axis from the side wall of the cylindrical recess of the hub body.
